(19) United States
(12) Patent Application Publication
(10) Pub. No.: US 2018/0353417 A1
(43) Pub. Date: Dec. 13, 2018

(54) Eyelid Cleansing and Care Kit

(71) Applicant: OCuSOFT, Inc., Rosenberg, TX (US)

(72) Inventors: Nat Adkins, JR., Richmond, TX (US); Troy Smith, Richmond, TX (US)

(21) Appl. No.: 16/108,438

(22) Filed: Aug. 22, 2018

Related U.S. Application Data
(63) Continuation-in-part of application No. 15/830,428, filed on Dec. 4, 2017, now Pat. No. 10,085,930.
Application No. 15/830,428 is a continuation of application No. 15/090,386, filed on Apr. 4, 2016, now Pat. No. 9,833,399.
(60) Provisional application No. 62/697,609, filed on Jul. 13, 2018.

(51) Int. Cl. A61K 8/68; A61K 9/00; A61K 33/20; A61K 8/04; A61K 8/60; A61K 8/86; A61K 8/42; A61Q 19/10; A61K 8/88; A61Q 19/00; A61K 8/55; A61K 8/44; A61K 8/34; A61K 47/14; A61K 47/10; A61K 47/02; A61K 8/20; A61K 8/49

(52) U.S. Cl. CPC A61K 8/68; A61K 2800/87; A61K 2800/524; A61K 9/0048; A61K 33/20; A61K 8/046; A61K 8/602; A61K 8/86; A61K 8/42; A61Q 19/10; A61K 8/88; A61Q 19/005; A61K 8/553; A61K 8/442; A61K 8/345; A61K 47/14; A61K 47/10; A61K 47/02; A61Q 19/007; A61K 2800/884; A61K 8/20; A61K 8/4973; A61K 2800/49

(57) ABSTRACT

A convenient all-in-one kit for cleansing the eyelids and treating bacterial blepharitis contains one or more fabric pads pre-moistened with a therapeutically effective amount of a hypochlorous acid-based composition. The kit can further include an eyelid cleanser composition as a kit component. The eyelid cleanser composition can be selected from a group consisting of a solution, a foam, a gel, a spray, a lotion, a suspension, an emulsion, an ointment and combinations thereof. The kit can further include one or more fabric pads or wipes impregnated/pre-moistened with the eyelid cleanser composition.

### CROSS-REFERENCE TO RELATED APPLICATIONS

The present application, pursuant to 35 U.S.C. 111(b), claims the benefit of provisional application Ser. No. 62/697,609 filed Jul. 13, 2018, and entitled “Eyelid Cleansing and Care Kit.” This application is also a continuation-in-part of and claims priority to Ser. No. 15/830,428 filed on Dec. 4, 2017, which is a continuation of U.S. Ser. No. 15/090,386 filed on Apr. 4, 2016, and now issued U.S. Pat. No. 9,833,399, the contents of which applications are incorporated by reference herein.

### BACKGROUND

The invention relates generally to kits and more particularly to an eyelid cleansing and care kit and methods for cleansing the eye and treating bacterial blepharitis.

Ocular health refers to eyes as well as structures associated with the eyes, eyelids for example. The eyelids are important in over-all ocular health because they protect the eyes from dangers such as approaching objects or from airborne contaminants, such as pollen, dust particles or other foreign bodies. The eyelids contain essential glands; the lacrimal glands and meibomian glands that produce layers of tear film that are critical for healthy eyes. When an individual blinks, a new tear film is created and tears are distributed across the cornea to lubricate the surface of the eye. This blinking action also “flushes” foreign materials from the eyes.

The eyelids, however, are subject to certain problems, which while very common, are none-the-less bothersome, especially for contact lens wearers, and may lead to other more serious complications. One complication is blepharitis. Blepharitis is a common chronic inflammation of the eyelids characterized by a scaly crust on the lid margins. The condition may be caused by a bacterial infection, or it may be allergic in origin or associated with seborrhea of the face and scalp. Treatment usually involves cleansing the eyelids on a regular basis to remove excess oil, debris, and desquamated skin that may be problematic.

Often associated with or secondary to blepharitis is a bacterial infection of the surface of the skin at the edge of the lid know as an internal hordeolum. Other such infections include external hordeolum, commonly referred to styes, which are infections of the tiny oil secreting meibomian glands along the edge of the eyelid, surrounding the eyelashes. A stye begins as a red, tender bump and usually fully develops within three days. Such conditions are accompanied by pain, redness and tenderness of the eyelid (or lid) margins. Although styes are often recurring, regular cleansing of the eyelid margins can minimize such conditions. A second problem is a chalazion, which is an inflammation of the meibomian glands inside the eyelid. Chalazia typically grow slowly over 2-3 weeks and although they do not typically cause pain, they often require surgical intervention if left untreated.

With any of the above-described problems, as well as other medical complications, such as rosacea and seborrhea, proper eyelid hygiene with the use of an eyelid cleanser may minimize the severity of the outbreak, or prevent the problem altogether if caught early. Eyelid cleansers are also used for cleaning eyelashes, eyelids or the periocular area and may be used as a pre-operative scrub to help reduce the presence of harmful bacteria which may cause infection, inflammation, or even endophthalmitis in patients.

### SUMMARY

According to an embodiment, a kit for maintaining eyelid hygiene consists essentially of one or more fabric pads, wherein each fabric pad is pre-moistened with a therapeutically effective amount of a hypochlorous acid-based composition.

According to another embodiment, a kit for maintaining eyelid hygiene includes: one or more fabric pads pre-moistened with a therapeutically effective amount of an eyelid cleanser composition; and one or more fabric pads pre-moistened with a therapeutically effective amount of a hypochlorous acid-based composition.

According to yet another embodiment, a kit for maintaining eyelid hygiene includes: one or more fabric pads pre-moistened with a therapeutically effective amount of a hypochlorous acid-based composition; and an eyelid cleanser composition.

In one embodiment, a method for treating bacterial blepharitis involves gently rubbing a fabric pad pre-moistened with a hypochlorous acid-based composition over an eyelid, wherein the hypochlorous acid-based composition includes ≥99% water and ≤0.01-0.02% hypochlorous acid. The method can further involve pre-cleansing the eyelid by applying a desired amount of an eyelid cleanser composition to the eyelid.

### DETAILED DESCRIPTION

The various embodiments of the invention include a convenient all-in-one eyelid cleansing and care kit that can improve compliance in treating bacterial blepharitis. According to an embodiment, the kit contains: (1) an eyelid cleanser composition; and/or (2) a hypochlorous acid-based composition.

### Eyelid Cleanser Composition

The eyelid cleanser composition can be any suitable composition that can clean the eyelids and eyes and remove excessive oil, debris, and desquamated skin from the eyelids.

In one embodiment, the eyelid cleanser composition includes a composition having: polyhexamethylene biguanide; 1,2-hexanediol; and 1,2-octanediol and one or more surfactants. The surfactants may be present in an amount effective to control a pH of the first composition within a desired pH range. U.S. Pat. No. 7,951,387, herein incorporated by reference in its entirety, describes such a composition having polyhexamethylene biguanide; 1,2-hexanediol; and 1,2-octanediol.

In another embodiment, the eyelid cleanser composition includes a composition having: purified water; PEG-80 sorbitan laurate; sodium trideceth sulfate; PEG-150 distearate; sodium lauroamphoacetate; cocamidopropyl hydroxysultaine; sodium laureth-13 carboxylate; sodium chloride; PEG-15 cocopolyamine; polyhexamethylene biguanide; potassium sorbate; 1,2 hexanediol; and Caprylyl glycol. U.S. Pat. No. 9,278,079, herein incorporated by reference in its entirety, describes such a composition.

In another embodiment, the eyelid cleanser composition includes a composition comprising: (a) a mixture of antimicrobial agents that substantially eliminates at least seven bacterial strains, wherein the mixture includes polyaminopropyl biguanide, a 1,2-glycol, and a sphingoid lipid; (b) a blended surfactant solution that maintains the first ophthalmic composition at a pH between 5.5 and 7.5, wherein the blended surfactant solution has a foaming ability; (c) a foam stabilizer; and (d) a moisturizer. U.S. Pat. No. 9,833,399, herein incorporated by reference in its entirety, describes such a composition.

The eyelid cleanser composition can be selected from a group consisting of a solution, a foam, a gel, a spray, a lotion, a suspension, an emulsion, an ointment and combinations thereof. The eyelid cleanser composition can be contained and dispensed from a suitable dispenser. In certain embodiments, the container is a pump dispenser configured to contain and deliver the eyelid cleanser composition as a pre-lathered foam

In certain embodiments, the kit can include one or more fabric pads or wipes impregnated/pre-moistened with a therapeutically effective amount of the eyelid cleanser composition. The term “pad” refers to a thick piece of fabric that is capable of holding or absorbing the eyelid cleanser composition. For example, the fabric can be a lint-free fabric, such as, rayon or another suitable material. In certain embodiments, the fabric pad may be a cotton pad. The pads may be single use disposable pads. The kit can include between 1-100 pads. The pads may be sealed or wrapped within suitable packaging.

In certain embodiments, the kit can include one or more single use dry pads. A desired amount of the eyelid cleanser composition may be then applied to a dry pad prior to using it to cleanse the eyelids.

In another embodiment, the kit can include a swab or another suitable applicator/system that can be pre-filled with a desired amount of the eyelid cleanser composition. U.S. Pat. Pub. No. 2015/0320594, herein incorporated by reference in its entirety, describes such an applicator/system.

In one embodiment, the eyelid cleanser kit includes packaging for a plurality of pre-moistened pads containing an embodiment of the eyelid cleanser composition discussed herein and a bottle of a hypochlorous acid-based composition in solution, spray or gel form.

### Hypochlorous Acid-based Composition

The eyelid cleanser composition is effective for daily use in all blepharitis conditions. However, in the most severe conditions, it may be used in combination with a hypochlorous acid-based composition to optimally treat severe cases of blepharitis. Since the hypochlorous acid-based composition contains hypochlorous acid but not a surfactant, it may not optimally dissolve and remove oil, debris and desquamated skin. Therefore, a combination therapy involving the eyelid cleanser and hypochlorous acid-based compositions may be effective in debriding the oil, scales and debris often associated with eyelid irritations. This combination therapy is fast acting against microorganisms and expedites wound healing of the eyelids while addressing inflammation and long term eyelid hygiene. In one embodiment, the hypochlorous acid-based composition contains ≥99% water and ≤0.01-0.02% hypochlorous acid. The hypochlorous acid-based compositions can be used to substantially eradicate bacteria on the outer eyelids and eyelid margins.

The hypochlorous acid-based composition can be selected from a group consisting of a solution, a foam, a gel, a spray, a lotion, a suspension, an emulsion, an ointment and combinations thereof.

In certain embodiments, the kit can include one or more fabric pads or wipes impregnated/pre-moistened with a therapeutically effective amount of the hypochlorous acid-based composition. As described earlier, the fabric can be a lint-free fabric, such as, rayon or another suitable material. In certain embodiments, the fabric pad may be a cotton pad. The pads may be single use disposable pads. The kit can include between 1-100 pads. The pads may be sealed or wrapped within suitable packaging.

In certain embodiments, the kit can include one or more single use dry pads. A desired amount of the eyelid cleanser composition may be then applied to a dry pad prior to using it.

In one embodiment, a method for manufacturing an eyelid cleansing kit involves packaging the eyelid cleanser composition as a kit component together with a hypochlorous acid-based composition.

In another embodiment, a method for cleansing the eyelids is disclosed. The method involves cleaning the eyelids by applying a desired or effective amount of the eyelid cleanser composition to each eyelid. The effective amount may generally include a small amount of the composition. The composition may be lightly rubbed on or around the affected area. Depending on the embodiment of the eyelid cleanser composition, it may be either rinsed off or left on the eyelids after application. This step can clean and remove excessive oil, debris and desquamated skin from the eyelids.

A desired amount of the hypochlorous acid-based composition is then applied to each eyelid. For example, a desired amount of the hypochlorous acid-based composition may be sprayed on to a dry pad. Alternately, a fabric pad pre-moistened with a therapeutically effective amount of the hypochlorous acid-based composition can be used. One such moistened pad may be used for each eyelid. The moistened pad is gently rubbed over the eyelids using a side-to-side motion. There is no need to rinse the eyelid afterwards. The pad may be disposed after use. The hypochlorous acid-based composition can remove microorganisms, such as, bacteria on the eyelids. It can also facilitate immediate care of minor irritations to the skin, cleansing minor cuts, minor scrapes and minor burns.

In one or more embodiments, for maximum effect and results, the user may apply the eyelid cleanser composition and the hypochlorous acid-based composition to the eyelids multiple times a day, for example, 2-3 times a day. Prior to (re)application, the user may be required to wash their hands, and clean and dry the eyelids.

The present invention can comprise or consist essentially of the components as well as other components which are not recited (such as, biological gloves). The terms “having,” “including,” and “comprised of” are also to be construed as open ended unless the context suggests otherwise.

As used herein, the term “therapeutically effective amount” of a composition refers to an amount sufficient to cleanse the eyelid and treat a bacterial infection. In some embodiments, a therapeutically effective amount of a composition is an amount that is sufficient, when administered to a subject suffering from or susceptible to an ocular disease, disorder, and/or condition, to treat, diagnose, prevent, and/or delay the onset of one or more symptoms of the disease, disorder, and/or condition. As will be appreciated by those of ordinary skill in this art, and effective amount of a substance may vary depending on such factors as the desired biological endpoint, the patient, etc. For example, the effective amount of a composition to treat bacterial blepharitis is the amount that alleviates, ameliorates, relieves, inhibits, prevents, delays onset of, reduces severity of and/or reduces incidence of one or more symptoms or features associated with the infection.

Those skilled in the art will recognize that the kits may optionally contain instructions for use of the kit components.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

We claim:

1. A kit for maintaining eyelid hygiene, wherein the kit consists essentially of one or more fabric pads, wherein each fabric pad is pre-moistened with a therapeutically effective amount of a hypochlorous acid-based composition.

2. The kit according to claim 1, wherein the hypochlorous acid-based composition includes ≥99% water and ≤0.01-0.02% hypochlorous acid.

3. The kit according to claim 1, wherein the fabric pad comprises a lint-free fabric.

4. The kit according to claim 1, wherein the fabric pad is rayon pad.

5. The kit according to claim 1, wherein the fabric pad is a cotton pad.

6. The kit according to claim 1, wherein the fabric pads are single use disposable pads.

7. The kit according to claim 1, wherein the kit contains from about one to about one hundred fabric pads.

8. A kit for maintaining eyelid hygiene, wherein the kit includes:
(a) one or more fabric pads pre-moistened with a therapeutically effective amount of an eyelid cleanser composition; and
(b) one or more fabric pads pre-moistened with a therapeutically effective amount of a hypochlorous acid-based composition.

9. The kit according to claim 8, wherein the eyelid cleanser composition includes: polyhexamethylene biguanide; 1,2-hexanediol; and 1,2-octanediol and one or more surfactants.

10. The kit according to claim 8, wherein the eyelid cleanser composition includes: PEG-80 sorbitan laurate; sodium trideceth sulfate; PEG-150 distearate; sodium lauroamphoacetate; cocamidopropyl hydroxysultaine; sodium laureth-13 carboxylate; PEG-15 cocopolyamine; polyhexamethylene biguanide; potassium sorbate; 1,2 hexanediol; and caprylyl glycol.

11. The kit according to claim 8, wherein the eyelid cleanser composition includes:
a) a mixture of antimicrobial agents that substantially eliminates at least seven bacterial strains, wherein the mixture includes polyaminopropyl biguanide, a 1,2-glycol, and a sphingoid lipid;
(b) a blended surfactant solution that maintains the first ophthalmic composition at a pH between 5.5 and 7.5, wherein the blended surfactant solution has a foaming ability;
(c) a foam stabilizer; and
(d) a moisturizer.

12. The kit according to claim 8, wherein the hypochlorous acid-based composition includes ≥99% water and ≤0.01-0.02% hypochlorous acid.

13. A kit for maintaining eyelid hygiene, wherein the kit includes:
(a) one or more fabric pads pre-moistened with a therapeutically effective amount of a hypochlorous acid-based composition; and
(b) an eyelid cleanser composition.

14. The kit according to claim 13, wherein the hypochlorous acid-based composition includes ≥99% water and ≤0.01-0.02% hypochlorous acid.

15. The kit according to claim 13, wherein the eyelid cleanser composition includes: polyhexamethylene biguanide; 1,2-hexanediol; and 1,2-octanediol and one or more surfactants.

16. The kit according to claim 13, wherein the eyelid cleanser composition includes: PEG-80 sorbitan laurate; sodium trideceth sulfate; PEG-150 distearate; sodium lauroamphoacetate; cocamidopropyl hydroxysultaine; sodium laureth-13 carboxylate; PEG-15 cocopolyamine; polyhexamethylene biguanide; potassium sorbate; 1,2 hexanediol; and caprylyl glycol.

17. The kit according to claim 13, wherein the eyelid cleanser composition includes:
a) a mixture of antimicrobial agents that substantially eliminates at least seven bacterial strains, wherein the mixture includes polyaminopropyl biguanide, a 1,2-glycol, and a sphingoid lipid;
(b) a blended surfactant solution that maintains the first ophthalmic composition at a pH between 5.5 and 7.5, wherein the blended surfactant solution has a foaming ability;
(c) a foam stabilizer; and
(d) a moisturizer.

18. The kit according to claim 13, wherein the eyelid cleanser composition is provided as a solution, a foam, a gel, a spray a lotion, a suspension, an emulsion an ointment, or combinations thereof.

19. The kit according to claim 13, further comprising one or more fabric pads.

20. A method for treating bacterial blepharitis, comprising:
gently rubbing a fabric pad pre-moistened with a hypochlorous acid-based composition over an eyelid,
wherein the hypochlorous acid-based composition includes ≥99% water and ≤0.01-0.02% hypochlorous acid.

21. The method according to claim 20, further comprising allowing the hypochlorous acid-based composition to remain on the eyelid.

22. The method according to claim 20, further comprising pre-cleansing the eyelid by applying a desired amount of an eyelid cleanser composition to the eyelid.

23. The method according to claim 20, further comprising repeating the rubbing of the eyelid with the hypochlorous acid-based composition multiple times a day.

24. The method according to claim 23, further comprising pre-cleansing the eyelid by applying a desired amount of an eyelid cleanser composition to the eyelid before each rubbing of the eyelid with the hypochlorous acid-based composition.
